(19) United States
(12) Patent Application Publication
(10) Pub. No.: US 2023/0158200 A1
(43) Pub. Date: May 25, 2023

(54) SYSTEM FOR PROTECTION AGAINST AIRBORNE PATHOGENS IN A SURGICAL ENVIRONMENT

(71) Applicant: Eberhard Karls Universität Tuebingen Medizinische Fakultät, Tuebingen (DE)

(72) Inventor: Marc Reymond, Tübingen (DE)

(21) Appl. No.: 18/155,648

(22) Filed: Jan. 17, 2023

Related U.S. Application Data
(63) Continuation of application No. PCT/EP21/69826, filed on Jul. 15, 2021.

(30) Foreign Application Priority Data
Jul. 16, 2020 (DE) 10 2020 118

(51) Int. Cl. A61L 9/22; A61L 9/20; A61L 9/03

(52) U.S. Cl. CPC A61L 9/22; A61L 9/20; A61L 9/03; A61L 2209/14

(57) ABSTRACT

The present invention relates to a system for protecting medical personnel and/or a patient from airborne pathogens in a surgical environment and a method for protecting medical personnel and/or a patient from airborne pathogens in a surgical environment.

### CROSS-REFERENCES TO RELATED APPLICATIONS

This application is a continuation of copending international patent application PCT/EP2021/069826 filed on 15 Jul. 2021 and designating the U.S., which has been published in German, and claims priority from German patent application DE 10 2020 118 863.3 filed on 16 Jul. 2020. The entire contents of these prior applications are incorporated herein by reference.

### FIELD OF THE INVENTION

The present invention relates to a system for protecting medical personnel and/or a patient from airborne pathogens in a surgical environment and a method for protecting medical personnel and/or a patient from airborne pathogens in a surgical environment.

### BACKGROUND OF THE INVENTION

During surgical procedures, medical personnel are exposed to special risks of infection. The procedures may result in the excretion of large quantities of pathogens if the patient is an infected person.

Contamination of medical personnel by liquids can be adequately prevented by protective clothing, gloves, and glasses. In contrast, the risk of infection from virus-containing aerosols appears to be very high. Previous work has demonstrated the presence of several pathogens in surgical smoke, including Corynebacterium, human papillomavirus (HPV), poliovirus, human immunodeficiency virus (HIV), and hepatitis viruses.

Airborne pathogens pose a special challenge. These are pathogenic particles or agents, such as viruses or bacteria, which are suspended in the room air for a long time, e.g., in an aerosol, and, unlike liquid droplets, do not immediately sink to the ground after being excreted, e.g., via breathing or the cough of an infected patient.

Airborne pathogens include SARS-CoV-2 (abbreviation for Severe Acute Respiratory Syndrome Coronavirus 2) By May 2020, according to the Robert Koch Institute (RKI), 10,000 healthcare professionals in Germany had been infected with SARS-CoV-2; 16 of them died of COVID-19, a disease caused by SARS-CoV-2.

The sources and pathways of SARS-CoV-2 contamination have not been elucidated in detail. However, there is no doubt that contacts with blood, feces, the gastrointestinal mucosa, peritoneal fluid play important roles. Also, SARS-CoV-2 is thought to be present in surgical smoke; Mowbray et al. (2020), Safe management of surgical smoke in the age of COVID-19, British Journal of Surgery, doi:101002/bjs.11679.

Current systems used in operating rooms (ORs) to protect medical personnel from infection, particularly airborne infection, provide inadequate protection. In particular, it is unclear how effectively current OR air filtration systems (according to ISO 14644-1 class≤5) can remove airborne particles the size of SARS-CoV-2 with a diameter of 60 to 140 nm.

Accordingly, there is an urgent need for systems and methods to mitigate risks to healthcare workers and the patient during surgical procedures due to airborne pathogens.

### SUMMARY OF THE INVENTION

The invention is therefore based on the task of providing a system and method for protecting medical personnel and/or a patient from airborne pathogens in a surgical environment, which prevents or at least reduces the risk of infection of the personnel and/or the patient compared to conventional systems.

This task is solved by a system comprising: a first electrode configured for attachment to the medical personnel,
a second electrode configured for attachment to the patient,
a first generator for applying voltage to the electrodes,
   wherein the first and/or second electrodes are configured to ionize airborne pathogens outside the patients body, and wherein the first and second electrodes have the same polarity when energized.
It is further solved by a method comprising the steps of:
1. Attaching a first electrode to the medical personnel,
2. Attaching a second electrode to the patient,
3. Applying voltage to the first and second electrodes such that the airborne pathogens are ionized outside the body of the patient, wherein the first and second electrodes have the same polarity, preferably negative polarity.

The method according to the invention is preferably carried out using the system according to the invention.

The inventors have realized that the technology of so-called electrostatic precipitation, which is basically known in the prior art, can be used in a targeted manner to minimize, and possibly even avoid, the risk of infection of medical personnel and/or the patient with airborne pathogens during a surgical procedure. For this purpose, voltages of the same polarity are generated using electrodes, e.g., brush electrodes, which are placed outside the body of the patient and the medical personnel. By means of the first or second electrode, the airborne pathogens outside the patients body are ionized and provided with a charge of such polarity that corresponds to the polarity of the charge generated by the electrodes on the medical personnel and/or the patient. According to the invention, when voltage is applied by the generator, the first and second electrodes have the same polarity, i.e., negative or positive polarity.

Due to the same charge or polarity of the ionized pathogens and medical personnel or patients, electrostatic repulsion prevents contamination with the pathogen.

The first electrode is attached to a suitable location on the medical personnel, e.g., on the chest (e.g., as or in a collar), head (e.g., on a helmet or cap), face (e.g., integrated into a protective mask), etc. The second electrode is attached to the patient at a suitable location, e.g., in the immediate vicinity of the surgical area and/or at or near the patient mouth and/or nose.

In one embodiment of the invention, the system may be a mobile system.

During electrostatic precipitation, charge carriers are released into a gas such as room air, usually electrons. This leads to the charging of particles in the gas in the electric field.

In the prior art, the electrostatic precipitation used in the invention is employed in so-called electrostatic precipitators, which serve to remove fine dust from exhaust gases, which are produced, for example, in the commercial manufacture of cement and paper. There, the charged dust particles are transported to a precipitation electrode, to which they adhere. The particles can be removed from the collecting electrode as a layer of dust.

In medicine, electrostatic precipitation is used to ionize particles in surgical smoke; see Mowbray et al. (op. cit.). Unlike the system according to the invention, negative ions are generated within the body, namely in the abdominal cavity. In the known system, these ions impart a temporary negative charge to the particles of surgical smoke. The electrostatically charged particles are attracted to the patient tissue due to the presence of the standard patient return electrode used during surgery. The charge is neutralized when the particles precipitate onto the surface of the peritoneal wall. A commercially available device used in the known system is known as Ultravision™, which is described in more detail in WO 2018/234803. However, the known system has the disadvantage that it is not suitable for protecting the personnel and/or the patient for operations in which no surgical smoke is generated, for example, because there is no exposure to heat. The known system also does not provide sufficient protection against airborne pathogens that are outside the body of the patient or medical personnel.

The invention provides a remedy for this problem. It provides protection for both medical personnel from airborne pathogens excreted by the patient or members of the surgical team, and for the patient from airborne pathogens excreted by the medical personnel.

According to one embodiment of the invention, the airborne pathogens are viruses, preferably SARS-CoV-2.

This measure has the advantage of adapting the system according to the invention to particularly relevant pandemically occurring pathogens. Particularly in pandemics, it is crucial for successfully combating the spread of the pathogen that the treating medical personnel are protected from infection. The system according to the invention thus represents an effective tool for containing pandemically occurring viral diseases.

According to another embodiment of the invention, the first and second electrodes have negative polarity when voltage is applied.

This measure has the advantage of being adapted to electrodes and systems used in the medical field, such as Ultravision™ (Alesi Surgical Cardiff, United Kingdom). The manufacture of the system according to the invention is thus facilitated and the costs reduced.

According to one embodiment of the invention, the first electrode is configured for attachment to or near the head of the medical personnel.

The measure has the advantage of generating charges in the area of the upper respiratory tract, i.e., the mouth and nose, the main entry points of airborne pathogens, which prevent the pathogens from approaching by electrostatic repulsion. This can be applied to the head or nearby, e.g., the neck.

It is understood that the second electrode can also be applied in the area of the head, corresponding to the first electrode. As a result, charge carriers with a polarity corresponding to that of the airborne pathogens are also formed on the patient, for example in the region of the mouth and/or nose. The uptake of the pathogens by the patient via the upper respiratory tract is thus effectively prevented.

According to a still further embodiment of the invention, the first electrode is integrated into a headband, cap, visor, and/or collar.

This measure has the advantage of creating the constructive conditions for attaching the first electrode to the head of the medical personnel.

According to a more valuable embodiment of the invention, the second electrode is configured for attachment to or in proximity to a surgical site on the patient.

This measure has the advantage of ionizing airborne pathogens excreted by the patient immediately at the site of their release and preventing uncontrolled airborne dissemination. According to the invention, placement at or near a surgical site requires a spacing small enough to ensure effective ionization of airborne pathogens excreted from the surgical site.

In another embodiment of the invention, the second electrode is integrated into a surgical drape, preferably in the region of the surgical opening.

This measure has the advantage of ensuring immediate ionization of airborne pathogens escaping from the surgical site (e.g., abdomen). So-called brush electrodes (“velcro” style), which can be sewn into a surgical drape, e.g., in the circumferential area of the surgical opening, are particularly suitable.

In a further embodiment, the system according to the invention comprises an aspirator for aspirating the airborne pathogens, in particular the ionized airborne pathogens.

This measure has the advantage of adding a component to the system according to the invention, which ensures the safe removal of airborne pathogens from the surgical site. A suitable aspirator or suction device, respectively, comprises the so-called frontal vortex suction. The aspirator can be suitably positioned in the system to achieve maximum clearance of the pathogens, for example, between the patient and the medical staff. The flow can be, for example, 300-500 m³/h per aspirator. The frontal vortex aspirator may be disposable and preferably sterile. In one embodiment, such a device may typically have dimensions of about 60×10 cm. The air velocity may be, in one embodiment, about 16 m/s. In another embodiment, the aspirator may be maintained under negative pressure until placed in hermetic containers, such as plastic bags with seals, for safe disposal. In one embodiment, the aspirator may be attachable to an operating table. Alternatively or additionally, the aspirator may be integrated into the surgical drape to facilitate and increase handling safety. In a further embodiment, the aspirator is a mobile device. In yet another embodiment, the aspirator is provided with a cover, preferably a circumferential cover, that minimizes air leakage into the environment.

In a further embodiment of the invention, the aspirator or suction device is configured for being supplied with voltage, preferably the voltage having a polarity opposite to the polarity of the first and/or second electrode.

This measure has the advantage that an additional deflection of the ionized airborne pathogens in the direction of the aspirator takes place by means of electrostatic interaction. The aspirator can be energized by the generator that also supplies the electrodes with voltage or by a separate second generator. According to one embodiment of the invention, the application of voltage to the aspirator can be effected via a third electrode which is designed for attachment to the aspirator. The third electrode can be connected to the first or a further generator.

In one embodiment, the method according to the invention therefore has the following further step:
4. Aspirating the ionized airborne pathogens, preferably via an aspirator which, when a voltage is applied to it, has a polarity which is opposite to the polarity of the first and/or second electrode.

In one embodiment of the invention, the system further comprises a device for emitting UV-C radiation.

In one embodiment of the invention, the system further comprises a device for emitting UV-C radiation.

This measure integrates an additional component that provides increased safety. Short-wave light with a wavelength of 250 nm to 270 nm provides activation energy to stimulate a photochemical reaction that has a cytotoxic effect on pathogens, especially viruses. UV-C illumination is a fast, inexpensive, comparatively low toxicity method. The device for emitting UV-C radiation is integrated in the aspirator system, for example. In a further embodiment, a certain number of UV-C light sources, typically 4 sources, optionally with a power of 160 W each, optionally with a wavelength of 254 nm, optionally ozone-free, are preferably arranged in longitudinal position in one, two or more cylindrical reactors to increase the exposure time to the UV-C light and the photodynamic toxicity.

In still another embodiment of the invention, the system further comprises a filter device, preferably a high efficiency particulate air (HEPA) filter and/or ultra low penetrating air (ULPA) filter.

This other component provides an additional decontamination effect. Ultra low penetrating or particle air (ULPA) filters retain 99.9% of 100 nm diameter particles, so they can capture most SARS-CoV-2 virus particles. The filter device is appropriately located in the system, such as in the ceiling of the operating room. The air flow can be adjusted as needed and is usually in the range of 10 m³/min and 50 m³/min.

This additional component also increases the safety and protection of personnel and patients from infection with airborne pathogens. In particular, it minimizes the risk of handling a contaminated filter device. The device for supplying disinfectant is suitably located in the system.

The system may further comprise other components, such as an activated carbon filter optionally placeable in and removable from an access opening of the system.

The features, embodiments and advantages of the system according to the invention apply accordingly to the method according to the invention.

Further advantages and features will be apparent from the following description of preferred embodiments and the accompanying drawings.

It is understood that the above features, which will be explained below, can be used not only in the combination indicated in each case, but also in other combinations or on their own, without departing from the scope of the present invention.

### BRIEF DESCRIPTION OF THE DRAWINGS

FIG. 1 shows an embodiment of the system according to the invention;

FIG. 2 shows a section of the embodiment shown in FIG. 1;

FIG. 3 shows an embodiment of the second electrode integrated into a surgical drape;

FIG. 4 shows embodiments of the first electrode for attachment to the body of medical personnel.

### DESCRIPTION OF PREFERRED EMBODIMENTS

In FIG. 1, the system according to the invention for the protection of medical personnel and/or a patient is indicated by reference numeral 10. The medical personnel is shown as M1, M2, M3 and the patient is shown as P. The system 10 comprises a first electrode 12 configured for attachment to the medical personnel M1, M2, M3, a second electrode 14 configured for attachment to the patient P, and a generator 16 for applying voltage to the electrodes 12, 14. In the illustrated embodiment, the second electrode 14 is attached to both the head region of the patient P and the surgical site 18. When voltage is applied to the electrodes 12, 14 via the generator 16, both electrodes 12, 14 have the same polarity, which is negative in the illustrated embodiment. Both electrodes 12, 14 thereby generate negatively charged ions in the immediate vicinity, for example in the vicinity of the face of the medical personnel M1, M2, M3, and on the patient P, there for example also in the vicinity of the face and/or the surgical site 18. At the surgical site 18, the negatively charged ions generated by the second electrode 14 impart a negative charge to the airborne pathogens, for example SARS-CoV-2, which may be released during the surgical procedure. Negative charges are represented by minus signs (−−−). Due to the negative charges of the airborne pathogens and the medical personnel M1, M2, M3, the airborne pathogens are kept away from the latter. This prevents infection of the medical personnel.

In the illustrated embodiment, an aspirator or suction device 20 is shown. This is connected to generator 16, which generates a positive polarity voltage at the aspirator device, represented by plus signs (+++). Aspirator 20 draws in ambient air and surgical smoke, including released airborne pathogens, which is facilitated by electrostatic interaction due to opposing charges. The airflow is shown by the thick arrows. The pathogens are thus removed from the surgical environment.

It is understood that the system according to the invention also prevents an infection of the patient P, should the medical personnel M1, M2, M3 excrete viruses. These would be negatively ionized by the first electrode 12 and, due to the negative charge on the patient P, kept away from the latter by electrostatic repulsion.

It is further understood that the charge ratios as shown in FIG. 1 may be reversed, i.e., positive charges on the medical personnel M1, M2, M3, the patient P and the surgical site 18 or the airborne pathogens, and negative charges on the suction device 20.

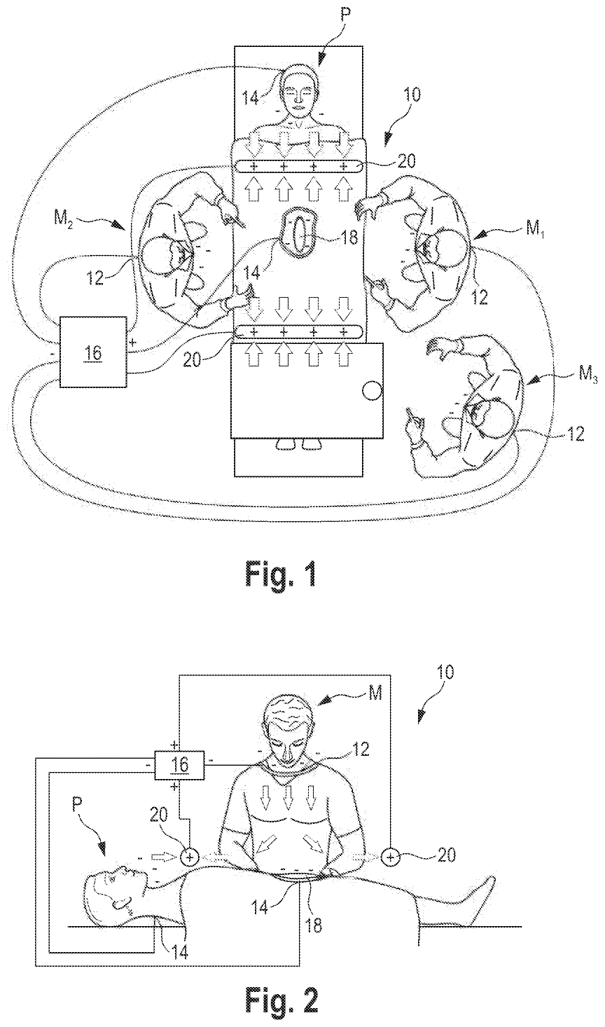

In FIG. 2, a section of the system 10 according to the invention for protecting medical personnel M and/or a patient P is shown. The first electrode 12 is placed in the head area of the patient P and at the surgical site 18, and is connected to the generator 16. The second electrode 14, which is also powered by the generator 16, is connected to the medical personnel M and is in the form of a collar. The viruses that may appear in the vicinity of the surgical site 18, as well as the surgical smoke, the area around the face of the medical personnel P and the area around the face of the patient P have negative charge carriers (−−). The aspirator 20 connected to the generator 16 is positively charged (+++). The negatively charged airborne pathogens are drawn into the latter due to the positive charge of the aspirator 20. The air flow is represented by the thick arrows.

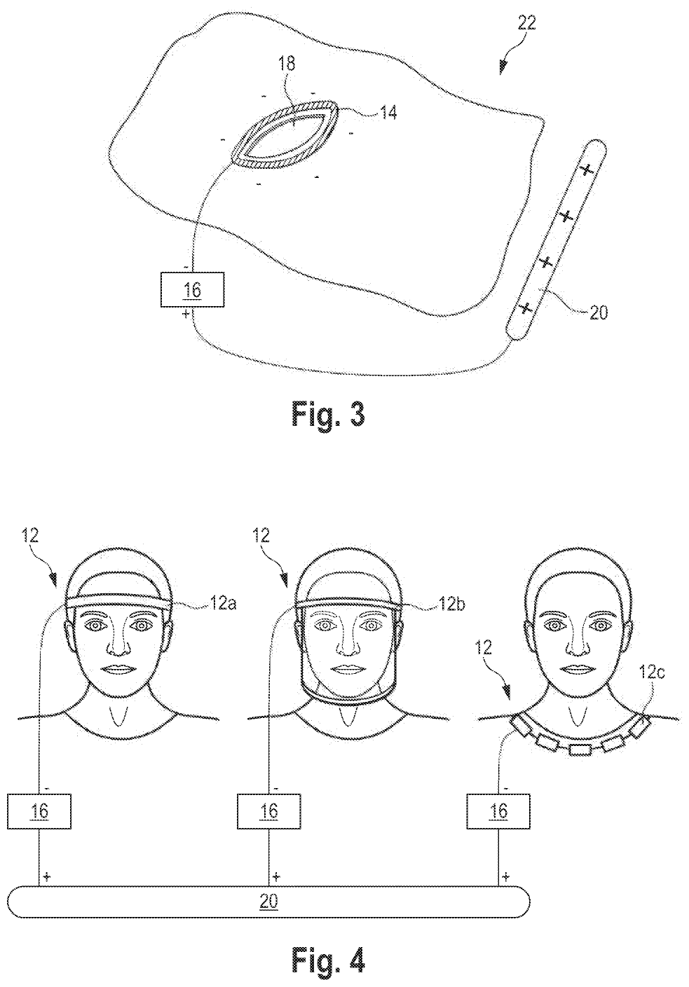

In FIG. 3, an embodiment of the second electrode 14 is shown in which the latter is integrated into a surgical drape or operating cloth 22, namely in an opening in the surgical drape 22, which is arranged over the operating site 18 in use. The second electrode is energized via the generator 16 and, in the illustrated embodiment, has negative polarity. Also shown is the aspirator 20, which is supplied with voltage via the generator 16 and has positive polarity in the variant shown.

In FIG. 4, various embodiments of the first electrode 12 are shown, namely in the form of a headband 12a, integrated into a face visor 12b, and in the form of a necklace 12c. Also shown are the generator 16 and the aspirator 20. The generator applies voltage to the first electrode 12, 12a, 12b, 12c so that the latter is negatively charged (−−−), and it applies voltage to the suction device so that the latter is positively charged (+++).

1. A system for protecting medical personnel and/or a patient from airborne pathogens in a surgical environment, comprising:
a first electrode configured for attachment to the medical personnel;
a second electrode configured for attachment to the patient; and
a first generator for applying voltage to the electrodes,
wherein the first and/or second electrode is configured to ionize the airborne pathogens outside the body of the patient, and wherein the first and second electrodes have the same polarity when voltage is applied.

2. The system of claim 1, wherein the airborne pathogens are viruses.

3. The system of claim 1, wherein the airborne pathogen is SARS-CoV-2.

4. The system of claim 1, wherein the first and second electrodes have negative polarity when voltage is applied.

5. The system of claim 1, wherein the first electrode is configured for attachment to or near the head of the medical personnel.

6. The system of claim 1, wherein the first electrode is integrated into a headband, cap, visor and/or collar.

7. The system of claim 1, wherein the second electrode is configured for attachment at or near a surgical site on the patient.

8. The system of claim 1, wherein the second electrode is integrated into a surgical drape.

9. The system of claim 1, wherein the second electrode is integrated into a surgical drape in the region of the surgical opening.

10. The system of claim 1, further comprising an aspirator for aspirating the ionized airborne pathogens.

11. The system of claim 10, wherein the aspirator is configured for being supplied with voltage.

12. The system of claim 11, wherein the aspirator, when voltage is applied to it, has a polarity opposite to the polarity of the first and/or second electrodes.

13. The system of claim 1, further comprising a device for emitting UV-C radiation.

14. The system of claim 1, further comprising a filter device.

15. The system of claim 14, wherein the filter device is a high efficiency particulate air (HEPA) filter and/or ultra low penetrating air (ULPA) filter.

16. The system of claim 1, further comprising a device for supplying disinfectant.

17. The system of claim 16, wherein the disinfectant is H₂O₂ and/or formaldehyde.

18. A method for protecting medical personnel and/or a patient from airborne pathogens in a surgical environment, comprising the following steps:
attaching a first electrode to the medical personnel;
attaching a second electrode to the patient;
attaching voltage to the first and second electrodes such that the airborne pathogens are ionized outside the body of the patient, wherein the first and second electrodes have the same polarity.

19. The method of claim 18, wherein the airborne pathogen is SARS-CoV-2.

20. The method of claim 18, wherein the first and second electrodes have negative polarity.

21. The method according to claim 18, further comprising aspirating the ionized airborne pathogens.

22. The method of claim 21, wherein the aspiration is carried out via an aspirator which, when voltage is applied to it, has a polarity opposite to the polarity of the first and/or second electrode.

23. The method of claim 18 carried out using the system according to claim 1.
